(19) United States
(12) Patent Application Publication
(10) Pub. No.: US 2018/0243475 A1
(43) Pub. Date: Aug. 30, 2018

(54) SILK FIBROIN CRYOGELS

(71) Applicant: Saint Louis University, St. Louis, MO (US)

(72) Inventors: Scott A. Sell, St. Louis, MO (US); Katherine R. Hixon, Davenport, IA (US); Era Jain, St. Louis, MO (US); Parin Kadakia, Vernon Hills, IL (US)

(21) Appl. No.: 15/757,213

(22) PCT Filed: Aug. 31, 2016
(86) PCT No.: PCT/US16/49678

§ 371 Date: Mar. 2, 2018

Related U.S. Application Data
(60) Provisional application No. 62/213,716, filed on Sep. 3, 2015.

(51) Int. Cl. A61L 27/36; A61L 27/56; A61L 27/38; A61L 27/54

(52) U.S. Cl. CPC A61L 27/3604; A61L 27/56; A61L 27/38; A61L 2430/02; A61L 2300/414; A61L 2300/252; A61L 27/54

(57) ABSTRACT

Disclosed are silk fibroin cryogels and methods for preparing silk fibroin cryogels. The silk fibroin cryogels can be used to promote cellular chemotaxis, enhance cell proliferation, enhance extracellular matrix production, promote calcified matrix production, and increase angiogenesis. The silk fibroin cryogels can further be used in the treatment of dermal wounds (burns, chronic wounds, etc.), bone tissue engineering and oral and maxillofacial repair.

### CROSS-REFERENCE TO RELATED APPLICATIONS

This application claims priority to U.S. Provisional Application No. 62/213,716, filed on Sep. 3, 2015, the disclosure of which is incorporated by reference in its entirety.

### BACKGROUND OF THE DISCLOSURE

The present disclosure relates generally to tissue engineering structures with biologically favorable structural and chemical properties. More particularly, the present disclosure relates to silk fibroin cryogels and methods for preparing silk fibroin cryogels. The silk fibroin cryogels can be used to promote cellular chemotaxis, enhance cell proliferation, enhance extracellular matrix production, promote calcified matrix production, and increase angiogenesis. The nature of the silk fibroin cryogels provides a template for cellular infiltration and guides tissue regeneration. The silk fibroin cryogels can be used in the treatment of dermal wounds (burns, chronic wounds, etc.), bone tissue engineering and oral and maxillofacial repair.

Cryogels provide a unique macroporous network that is ideal for promoting cellular attachment and infiltration, as well as native tissue ingrowth. While hydrogels are similar in chemical structure, their formation at room temperature leaves the primarily water-filled structure mechanically unstable. With cryogels, a polymer or monomer solution is frozen in a controlled manner such that ice crystal formation occurs throughout the gel prior to polymerization. When slowly thawed at a controlled temperature, these ice crystals melt leaving a macroporous structure that is ideal for cellular infiltration. Additionally, this particular method of formation leaves the resulting polymer structure with increased mechanical stability and a sponge-like consistency.

Silk fibroin has been long established as a material of interest for a number of medical applications due to its potential for cellular interaction, mechanical stability, and known rate of biodegradation. Silk fibroin has been used medicinally for centuries and continues to be of interest in the fields of tissue engineering and regenerative medicine. Silk fibroin has also been used to produce silk hydrogels.

Bone as a whole is completely dynamic, where osteoblasts create new bone tissue and osteoclasts break down old tissue. Under natural conditions, bone regeneration following a typical fracture begins healing through the formation of a hematoma. Angiogenesis occurs and mesenchymal stem cells infiltrate the area, leading to the differentiation of chondrocytes, osteoblasts, and osteoclasts to dynamically heal the injured bone. Initially, a soft tissue callus forms for structural support until the osteoblasts start producing new bone in its place. There are cases in which this natural fracture healing is not sufficient for regenerating the injured bone. Particularly, cases including traumatic fracture, osteosarcoma, congenital malformation, vehicular accident, or military blast wounds can create problematic bone defects. Injuries such as these produce what is known as a critical size defect; that is, a defect so large that it is incapable of naturally healing during the patient's lifetime. Clinically, any bone injury in which the defect site is twice the size of the injured bone's diameter falls into that category. If left to spontaneously heal, the injury site fills with soft tissue callus without the replacement with new bone, leading to nonunion.

The current treatment method for a critical size defect involves the use of a bone graft. Existing options for bone grafts include autografts, allografts, xenografts, and synthetic grafts. While autologous bone grafts are currently the favored choice due to their osteoconductive, osteoinductive, and osteogenic properties, and bone regeneration capability, there is a major complication rate of 8.6% involved in this procedure and the patient experiences major discomfort. Further, allografts come with high costs, possible infection, and lack of donor availability. While xenografts are not as commonly used, they offer an inexpensive alternative, but the results are not as successful.

Accordingly, there is a major need for a bone graft substitute that can treat these critical size defects while still remaining at a low cost for the patient. To create an ideal bone graft substitute for regenerating bone, the scaffold should possess osteoconductive, osteoinductive, and osteogenic properties. Hydrogels are conventionally a very common scaffold, but the mechanical integrity and nanoporous structure of hydrogels are not advantageous for this application. It would be advantageous if the alternative bone graft substitute structure allowed for a macroporous structure to support cellular infiltration and tissue regeneration. It would be further advantageous if the structure was biodegradable in nature such to allow controlled degradation and integration with host tissue.

### BRIEF DESCRIPTION OF THE DISCLOSURE

The present disclosure is generally directed to methods for preparing cryogel structures for tissue engineering and regenerative medicine. More particularly, the present disclosure is directed to methods for preparing cryogels from silk fibroin protein.

In one aspect, the present disclosure is directed to a composition comprising a silk fibroin cryogel.

In one aspect, the present disclosure is directed to a method for preparing a silk fibroin cryogel. The method comprises isolating silk fibroin protein to prepare a silk fibroin protein solution; sonicating the silk fibroin protein solution; and subjecting the sonicated silk fibroin protein solution to at least one freeze-thaw cycle.

In accordance with the present disclosure, compositions and methods have been discovered that provide a unique combination of cryogel structure and silk fibroin. The methods of the present disclosure have a broad and significant impact, as they provide an ideal scaffolding for a variety of tissue engineering applications. The silk fibroin cryogels further exhibit enhanced mechanical stability compared to silk fibroin hydrogels.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 is a schematic depicting the silk fibroin cryogel fabrication procedure.

FIG. 2 is a scanning electron micrograph (SEM) image of dehydrated silk cryogel at 100× magnification. Scale bar 200 μm.

FIG. 3 is a SEM image of dehydrated silk hydrogel at 100× magnification. Scale bar 200 μm.

FIGS. 4A-4F are SEM images of taken at 500× of (FIG. 4A) a CG hydrogel and (FIG. 4B) cryogel, (FIG. 4C) a NVP hydrogel and (FIG. 4D) cryogel, and (FIG. 4E) a SF hydrogel and (FIG. 4F) cryogel. Scale bar 100 μm.

FIG. 5 is a graph depicting average pore diameter of silk cryogels and silk hydrogels determined by ImageJ analysis of SEM images.

FIGS. 6A & 6B are graphs depicting the pore diameter (μm) (FIG. 6A) and pore area (μm²) (FIG. 6B) for CG, NVP, and SF cryogels.

FIGS. 7A-7F are μCT 3D reconstruction images of CG cryogels (FIG. 7A) and SF cryogels (FIG. 7B). A sagittal cross section of CG and SF cryogels displays the inner pores for CG cryogels (FIG. 7C) and SF cryogels (FIG. 7D), and the shading bars denote the size of the pores within the scaffold for CG cryogels (FIG. 7E) and SF cryogels (FIG. 7F). Scale bar 1 nm.

FIGS. 8A-8D are graphs depicting μCT scans of CG cryogels and SF cryogels at a threshold of 80 demonstrating the amount of the total volume of the cryogel that is filled with scaffold (FIG. 8A) and providing the overall connection density of the spaces (FIG. 8B). FIG. 8C is a graph depicting the average pore diameters (μm) of CG cryogels and SF cryogels. FIG. 8D is a graph depicting the heterogeneity of the pores.

FIGS. 9A-9D are graphs depicting mercury porosimetry performed on dehydrated cryogel types showing the average pore diameter (μm) (FIG. 9A), the total pore volume (mm³/g) (FIG. 9B), the total pore surface area (m²/g) (FIG. 9C), and the average pore size (um) of all three types of cryogels (FIG. 9D).

FIGS. 10A-10D are graphs depicting mercury porosimetry performed on hydrated cryogel types showing the average pore diameter (μm) (FIG. 10A), the total pore volume (mm³/g) (FIG. 10B), the total pore surface area (m²/g) (FIG. 10C), and the average pore size (um) of all three types of cryogels (FIG. 10D).

FIGS. 11A-11D are graphs depicting the swelling of dehydrated cryogels and hydrogels. FIG. 11A depicts the CG average swelling ratio (%) of cryogels vs. hydrogels. FIG. 11B depicts the NVP average swelling ratio (%) of cryogels vs. hydrogels. FIG. 11C depicts the SF average swelling ratio (%) of cryogels vs. hydrogels. FIG. 11D depicts the swelling ratio (%) of all three types of cryogels.

FIGS. 12A-12 are graphs depicting the ultimate compression of both cryogels and hydrogels for every material type. FIG. 12A depicts the average peak stress (kPa) at 50% compression. FIG. 12B depicts the average modulus (kPa) at 50% compression. FIG. 12C depicts the average peak stress (kPa) at 80% compression. FIG. 12D depicts the average modulus (kPa) at 80% compression.

FIGS. 13A-13C are graphs depicting the percent stress-relaxation over 28 days of cryogels vs. hydrogels for CG cryogels (FIG. 13A), NVP cryogels (FIG. 13B), and SF cryogels (FIG. 13C).

FIGS. 14A-14C are graphs depicting the hysteresis over 28 days of cryogels vs. hydrogels for CG (FIG. 14A), NVP (FIG. 14B), and SF (FIG. 14C).

FIGS. 15A-15D are graphs depicting the absorbance (mineralization) of cryogels over 21 days for CG (FIG. 15A), NVP (FIG. 15B), SF (FIG. 15C), and the fold-increase of all cryogels over controls (FIG. 15D).

FIGS. 16A & 16B are graphs depicting the peak stress (kPa) (FIG. 16A) and the modulus (kPa) (FIG. 16B) for all types of cryogels on days 7, 14, and 21 after mineralization.

FIGS. 17A-17L are SEM images taken at 500X of a plain CG cryogel (control) (FIG. 17A), Day 7 mineralized CG cryogel (FIG. 17B), Day 14 mineralized CG cryogel (FIG. 17C), Day 21 mineralized CG cryogel (FIG. 17D), plain NVP cryogel (control) (FIG. 17E), Day 7 mineralized NVP cryogel (FIG. 17F), Day 14 mineralized NVP cryogel (FIG. 17G), Day 21 mineralized NVP cryogel (FIG. 17H), plain SF cryogel (control) (FIG. 17I), Day 7 mineralized SF cryogel (FIG. 17J), Day 14 mineralized SF cryogel (FIG. 17K), and Day 21 mineralized SF cryogel (FIG. 17L). Scale bar 100 μm.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In accordance with the present disclosure, compositions and methods have been discovered that provide a unique combination of cryogel structure and silk fibroin. The silk fibroin cryogels further exhibit enhanced mechanical stability compared to silk fibroin hydrogels. The methods of the present disclosure have a broad and significant impact, as they provide an ideal scaffolding for a variety of tissue engineering applications. The silk fibroin cryogel can further include at least one biomolecule. The silk fibroin cryogel can be used to promote cellular chemotaxis, enhance cell proliferation, enhance extracellular matrix production, promote calcified matrix production, increase angiogenesis, and provide antimicrobial activity. The nature of the silk fibroin cryogel provides a template for cellular infiltration and can guide tissue regeneration. The silk fibroin cryogel can be used in the treatment of dermal wounds (burns, chronic wounds, etc.) or as a tissue engineering scaffold in a wide range of applications such as for bone engineering and oral and maxillofacial repair.

In one aspect, the present disclosure is directed to a composition including a silk fibroin cryogel. The silk fibroin cryogel is prepared using sonication as a physical trigger to induce β-sheet formation, followed by the silk fibroin solution being frozen in a controlled manner such that ice crystal formation occurs throughout the gel prior to polymerization. When slowly thawed at a controlled temperature, these ice crystals melt leaving a macroporous structure ideal for cellular infiltration. This particular method of formation leaves the resulting silk fibroin cryogel with a structure that possesses an increased mechanical stability and a sponge-like consistency. Structurally, the silk fibroin cryogels offer an ideal pore size, distribution, and interconnectivity for tissue engineering.

Silk fibroin protein advantageously is biodegradable (also referred to herein as “bioresorbable”). “Bioresorbable” and “biodegradable” are used interchangeably herein to refer to a material that is biocompatible as well as degradable and/or absorbable by a subject. Biodegradable material is intended to be broken down (usually gradually) by the body of a subject e.g., an animal, and in particular, a mammal. A bioresorbable material is intended to be absorbed or resorbed by the body of a subject, such that it eventually becomes essentially non-detectable at the site of application.

The silk fibroin cryogel can include from about 1% (w/v) to about 10% (w/v) silk fibroin protein. A particularly suitable concentration of silk fibroin in the silk fibroin cryogels is about 4.5% (w/v).

The silk fibroin cryogel can have a pore diameter ranging from about 15 μm to about 500 μm. Pore diameter can be determined by measuring images from scanning electron micrographs, for example. Particularly suitable methods for measuring pore size can be, for example, analysis of scanning electron micrographs using ImageJ software, μCT, and mercury intrusion porosimetry.

The silk fibroin cryogel can have a pore area ranging from about 2,000 μm² to about 20,000 μm². Pore area can be determined by measuring images from scanning electron micrographs. Particularly suitable methods for measuring pore area can be, for example, analysis of scanning electron micrographs using ImageJ software, μCT, and mercury intrusion porosimetry.

Dehydrated silk fibroin cryogel can have a total pore volume ranging from about 2,000 mm³/g to about 4,000 mm³/g. A particularly suitable method for determining silk fibroin cryogel porosity is by mercury intrusion porosimetry.

Dehydrated silk fibroin cryogel can have a total pore surface area of about 1 m²/g to about 1.7 m²/g. A particularly suitable method for determining silk fibroin cryogel pore area is by mercury intrusion porosimetry.

Hydrated silk fibroin cryogel can have a total pore volume ranging from about 600 mm³/g to about 1,000 mm³/g. A particularly suitable method for determining silk fibroin cryogel average pore diameter is by mercury intrusion porosimetry.

Hydrated silk fibroin cryogel can have a total pore surface area ranging from about 0.2 m²/g to about 0.27 m²/g. A particularly suitable method for determining silk fibroin cryogel total pore surface area is by mercury intrusion porosimetry.

Hydrated silk fibroin cryogel can have an average pore size ranging from 5 μm to about 40 μm. A particularly suitable method for determining silk fibroin cryogel average pore size is by mercury intrusion porosimetry.

Silk fibroin cryogels can have an average peak stress at 50% compression ranging from about 30 kPa to about 43 kPa. Silk fibroin cryogels can have an average peak stress at 80% compression ranging from about 25 kPa to about 110 kPa. Silk fibroin cryogels can have an average modulus at 50% compression ranging from about 100 kPa to about 150 kPa. Silk fibroin cryogels can have an average modulus at 80% compression ranging from about 10 kPa to about 200 kPa.

Silk fibroin cryogels can further include at least one additive. A particularly suitable additive is Manuka honey. Honey can be added to the aqueous silk fibroin solution. Preferably, honey is added to the aqueous silk fibroin solution prior to the sonication step.

Other particularly suitable additives include osteoinductive agents and osteoconductive agents. Particularly suitable osteoinductive agents and osteoconductive agents include, for example, bone char, hydroxyapatite, phosphates, calcium, carbonate, and combinations thereof. Osteoinductive agents and osteoconductive agents can be added to the aqueous silk fibroin solution. Preferably, the agent is added to the aqueous silk fibroin solution prior to the sonication step.

Other particularly suitable additives can be biomolecules. Suitable biomolecules can be, for example, growth factors, cytokines, bioactive lipids, immunoglobulins, and combinations thereof. Particularly suitable biomolecules can be, for example, platelet derived growth factor (PDGF), transforming growth factor beta (TGFβ), vascular endothelial growth factor (VEGF), fibroblast growth factor (FGF), epidermal growth factor (EGF), human growth factor (HGF), bone morphogenetic proteins (BMPs; e.g., BMP1, BMP2, BMP3, BMP4, BMPS, BMP6, BMP7, and BMP8a), insulin-like growth factors (e.g., IGF-1 and IGF-2), keratinocyte growth factor, connective tissue growth factor, chemotactic proteins, sphingosine 1-phosphate (S1P), various macrophage and monocyte mediators such as RANTES (Regulated upon Activation, Normal T-cell Expressed, and Secreted), tumor necrosis factor a (TNF α), interferon gamma (IFNγ), and granulocyte-macrophage colony stimulating factor (GM-CSF), lipoxin and combinations thereof. Suitable cytokines can be, for example, interleukins (e.g., IL-1-IL-36) and interferons (e.g., interferon type I, interferon type II, interferon type III).

The biomolecule can also be a preparation rich in growth factors (PRGF). PRGF can be prepared from blood or platelet rich plasma (PRP). To prepare PRGF, blood can be used to create PRP using methods known to those skilled in the art. For example, the HARVEST® SMARTPREP® 2 kit (Harvest Technologies Corp., Plymouth, MA) is a commercially available centrifugation system to create PRP. After obtaining PRP, the PRP is then subjected to a freeze-thaw-freeze (FTF) cycle for cell lysis. The FTF cycle can be performed by placing PRP in a −70° C. freezer, followed by thawing in a 37° C. water bath, and then returned to the −70° C. freezer. The frozen PRP is then lyophilized to create a dry PRGF powder that can be finely ground in a mortar and pestle prior to use.

In another aspect, the silk fibroin cryogel can include a plurality of cells. Cell types that can be used are, for example, endothelial cells, macrophages, adipose-derived stem cells, mesenchymal stem cells, embryonic stem cells, ligament fibroblasts, tendon fibroblasts, muscle fibroblasts, dermal fibroblasts, muscle cells and combinations thereof. The cells can be autologous cells, allogeneic cells, or xenogeneic cells. “Autologous cells” refer to cells that are donated and received by the same subject. For example, cells are obtained from subject A, incorporated into the scaffold support, and the cell-laden scaffold support can be implanted into subject A. “Allogeneic cells” refer to cells that are donated by a subject that is different from the recipient subject; however, the donor subject and recipient subject are from the same species. For example, cells are obtained from subject A, incorporated into the scaffold support, and the cell-laden scaffold support is implanted into subject B. “Xenogeneic cells” refer to cells that are obtained from or donated by a species that is different than the recipient. For example, cells are obtained from species A, incorporated into the scaffold support, and the cell-laden scaffold support is implanted into species B.

In another aspect, the silk fibroin cryogel can further be coated with a cell adhesion molecule. The cell adhesion molecule coating the silk fibroin cryogel would make contact with the silk fibroin protein making up the silk fibroin cryogel. The cell adhesion molecule can be, for example, fibronectin, vitronectin, collagen, RGD (arginine-glycine-aspartic acid) peptide, LDV (leucine-aspartic acid-valine) peptide, laminin and combinations thereof. Unique physical characteristics of silk fibroin cryogel enhance adsorption of cell adhesion molecules, induce favorable cell to extracellular matrix interactions, promote in vivo-like three-dimensional adhesion, activate cell signaling pathways, maintain cell phenotype, and support cell differentiation.

In another aspect, the silk fibroin cryogel can further be coated with other molecules such as, for example, recombinant and chemically synthesized proteins and peptides and nucleic acids (DNA and RNA).

In another aspect, the silk fibroin cryogel can further be mineralized by incubating the silk fibroin cryogel in a simulated body fluid for a desired time. Simulated body fluid is described in Oyane et al. (J. Biomed. Mater. 2003, 65A(2):188-195).

In another aspect, the present disclosure is directed to a method for preparing a silk fibroin cryogel. The method comprises isolating silk fibroin protein to prepare a silk fibroin protein solution; sonicating the silk fibroin protein solution; and subjecting the sonicated silk fibroin protein solution to at least one freeze-thaw cycle.

A particularly suitable method for isolating silk fibroin protein includes boiling a silk source in sodium carbonate for about 30 minutes. After boiling, the silk is washed in water. After washing, the water is drained and the silk is dried. Dried silk is then dissolved in a solution of lithium bromide at 60° C. for four hours and then at room temperature overnight. The dissolved silk solution is then dialyzed against water. After dialysis, the silk solution is preferably centrifuged twice at 8,500 RPM for 20 minutes.

Any suitable silk source can be used. A particularly suitable silk source is Mulberry silk from Bombyx mori silkworm silk cocoons. Other suitable silk sources include, for example, Tasar silk, Eri silk, Muga silk, Anaphe silk, Fagara silk, Coan silk, Mussel silk, and spider silk, and combinations thereof.

To prepare silk cryogels, the silk solution is transferred to a centrifuge tube, which is placed in a beaker of ice water, and sonicated for 30 seconds at a probe intensity setting of 2 (Fisher Sonic Dismembrator Model 100). After sonicating, the centrifuge tube is capped and placed in a methanol bath (−20° C.) for 24 hours. Following the 24 hours, the tubes are thawed in a water bath for 24 hours.

The methods of the present disclosure may be used to prepare silk fibroin cryogels. Advantageously, the silk fibroin cryogels provide a natural and thus, biocompatible cryogel containing cell attachment sites. The silk fibroin cryogels are suitable for tissue engineering and tissue regeneration. The silk fibroin cryogels are particularly suitable for bone regeneration and repair.

The disclosure will be more fully understood upon consideration of the following non-limiting Examples.

### EXAMPLES

The following materials were utilized throughout experimentation: Bombyx mori cocoons (The Yarn Tree, Ashville, NC), methanol (Fisher Scientific, Pittsburgh, Pa.), dialysis tubing (3.5 kD MWCO, SPECTRA/POR®, Spectrum Laboratories, Rancho Dominguez, Calif.), sodium carbonate (Acros), lithium bromide (Fisher), poly(ethylene glycol) (10000 g/mol, Alfa Aesar), phosphate buffered saline (10×, Hyclone), Manuka honey (Derma Sciences), bone char pellets (ground into grains that were less than 38 μm in diameter, Charcoal House).

Solutions of silk fibroin (SF) were prepared by cutting silk cocoons into four small pieces and discarding dead silk worms. 5 grams (g) of sliced cocoons and 4.24 g of Na₂CO₃ were added to 2 L of boiling distilled water for 30 minutes to remove the sericin protein of silk. Next, boiled silk cocoons underwent three 20 minute rinses with 1 L distilled water and then were dried at room temperature. Dried silk fibers were subsequently dissolved in a 9.3 M LiBr solution at 60° C. for 4 hours. The dissolved fibers were then dialyzed (3.5 kD MWCO, SPECTRA/POR®, Spectrum Laboratories, Rancho Dominguez, Calif.) against 1 L of distilled water at 4° C. for three days, with the water being changed every hour for the first 4 hours, twice the second day (morning and evening), and once the third day (morning). To eliminate any impurities, these aqueous solutions were centrifuged twice at 8500 rpm for 20 minutes. Final silk solutions were stored at 4° C. and utilized within two weeks of fabrication. Silk fibroin concentration was determined by drying out a known volume of silk and was found to be approximately 4% (w/v) following dialysis. To increase the silk concentration, solutions were dialyzed (3.5 kD MWCO, SPECTRA/POR®, Spectrum Laboratories, Rancho Dominguez, Calf.) against a 10% (w/v) PEG (10,000 g/mol) solution for 3 to 4 hours.

### Example 1

In this Example, Silk Fibroin Cryogels were prepared.

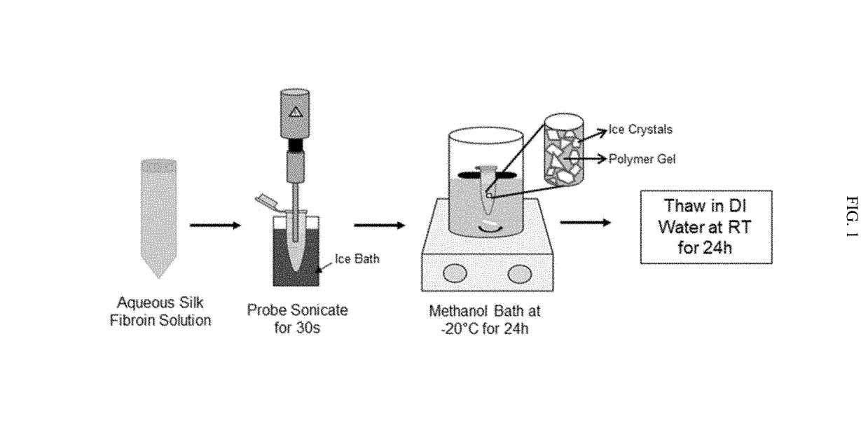

SF cryogels were prepared by adding 500 μL of silk solution (using a 4.5% (w/v) SF solution) to 2 mL centrifuge tubes. Holding the tubes steady in a small ice bath, silk solutions were probe sonicated with a Fisher Sonic Dismembrator Model 100 for 30 seconds at a probe intensity setting of 2 (Fisher Sonic Dismembrator Model 100). Following sonication, the tubes were stored in a methanol bath at −20° C. for 24 hours. The resulting cryogels were thawed in distilled water for 24 hours at room temperature before use (FIG. 1). For comparison, SF hydrogels were made with a similar process except these scaffolds were stored at room temperature for 24 hours instead of −20° C. The concentration of SF solution used to make these cryogels was 4.5% (w/v).

### Example 2

In this Example, SF cryogel fabrication was analyzed using different sonication parameters.

SF solutions were sonicated at 15 seconds, 30 seconds, and 60 seconds at a probe intensity of 2. A probe intensity of 2 was chosen arbitrarily to represent a low probe intensity. These gels were visually analyzed for their sol-gel transition activity (n=3). SF cryogels were made at probe intensities of 1, 2, and 3. Once again, these gels were visually examined for their sol-gel transition activity (n=3).

As summarized in Table 1, the resulting 15 seconds and 60 seconds cryogels biphasically separated into two layers, one clear and one white, whereas the 30 seconds cryogels remained a homogeneous white layer. The 15 seconds and 60 seconds cryogels encompassed inconsistent structures and the silk solutions that underwent 60 seconds of sonication gelled prior to the freezing step, which rendered the cryogelation step ineffective. Based on these visual results, 30 seconds of sonication time was subsequently used for SF cryogel preparation.

TABLE 1
Observation of SF cryogels with varying sonication times
(at probe intensity = 2).

| Sonication Times | Visual Observation |
|---|---|
| 15 s | Two separated layers (biphasic) |
| 30 s | Single layer (monophasic) |
| 60 s | Two separated layers (biphasic), |
| | gelled prior to freezing |

Table 2 summarizes the visual observations of the sol-gel transition activity for SF cryogels at probe intensities of 1, 2, and 3 at 30 seconds of sonication. With a probe intensity of 1, the resulting cryogels thawed completely to a liquid. At a probe intensity of 3, the silk solutions gelled prior to the freezing step, once again rendering the cryogelation step ineffective. These visual results, combined with the ones above, demonstrated that the optimal sonication time was 30 seconds and that the optimal probe intensity was 2.

TABLE 2
Observation of SF cryogels with varying probe intensities
(sonication time = 30 seconds).

| Probe Intensity | Visual Observation |
|---|---|
| 1 | Returned to a liquid upon thawing |
| 2 | Single layer (monophasic) |
| 3 | Single layer (monophasic), |
| | gelled prior to freezing |

### Example 3

In this Example, scanning electron microscopy was used to observe cross-sectional and surface morphology of SF cryogels.

Specifically, dehydrated SF cryogels and SF hydrogels (prepared as described in Example 1) were sputter coated with gold for 360 seconds using a Baltec SCD 005 sputter coater and imaged with a Zeiss EVO LS15 scanning electron microscope at an operating voltage of 10 kV. Pore diameter measurements were completed with ImageJ on 60 random pores per condition. For comparison to other cryogels, chitosan gelatin (CG) cryogels and N-Vinyl-2-pyrrolidone (NVP) cryogels were prepared.

To prepare CG cryogels, a 10 mL solution of 1% acetic acid (Fisher Scientific, N.J.) was prepared. 80 mg of low viscosity chitosan (MP Biomedicals, Ohio) was ultraviolet (UV) sterilized and dissolved in 8 mL of the 1% acetic acid solution. The solution was placed on a mechanical spinner until thoroughly mixed. 320 mg of gelatin from cold water fish skin (Sigma-Aldrich, St. Louis, Mo.) was UV sterilized and added to the chitosan solution. To avoid the formation of bubbles, the vial was placed on a mechanical shaker for approximately one hour until the gelatin was completely dissolved. The remaining 2 mL of 1% acetic acid was combined with glutaraldehyde (Sigma-Aldrich) to create a 1% glutaraldehyde solution. Both vials were placed at 4° C. for one hour. The solutions were mixed by slowly pouring between the vials and then poured into pre-cooled (−20° C.) 3 cc syringes (BD, New Jersey). PARAFILM (Bemis, Wisconsin) was used to seal off either side of the syringe and filled syringes were immediately placed in a −20° C. methanol (Fisher Scientific, New Jersey) bath. After at least 16 hours, the CG cryogels were taken out, the PARAFILM removed, and the gel filled syringes placed in room temperature, sterile water until thawed. To create the corresponding hydrogel, the previous procedure was followed and the polymer solution was placed at room temperature for 16 hours to ensure complete formation.

To prepare NVP cryogels, 7 mL of deionizing (DI) water was combined with 500 μL of N-Vinyl-2-pyrrolidone (NVP) (Acros, New Jersey) in a 50 mL tube (Fisher Scientific, New Jersey). Once mixed, 0.15 g of Bis-Acrylamide (Promega, Madison, Wis.) was added and the total volume brought up to 10 mL with additional DI water. This mixture underwent a freeze/thaw cycle between −20° C. and 4° C., respectively. The cycle began with a freeze of 30 minutes, thaw of 15 minutes, freeze of 30 minutes, thaw of 10 minutes, freeze of one hour, and complete thaw. Once melted, the solution was purged with Argon for two minutes. Polymerization was initiated by the addition of 20 μL of TEMED and a premade solution of 10 mg Ammonium Persulfate (APS) (Acros, New Jersey) in 100 μL DI water. The solution was vortexed between additions of these additives and then poured into pre-cooled (-20° C.) 3 cc syringes. PARAFILM was used to seal off either side of the syringe and filled syringes were immediately placed in a −20° C. methanol bath. After at least 16 hours, the cryogels were removed, the PARAFILM removed, and the gel filled syringes placed in room temperature water until thawed. To create the corresponding hydrogel, the previous procedure was followed and the polymer solution was placed at room temperature for 16 hours to ensure complete formation.

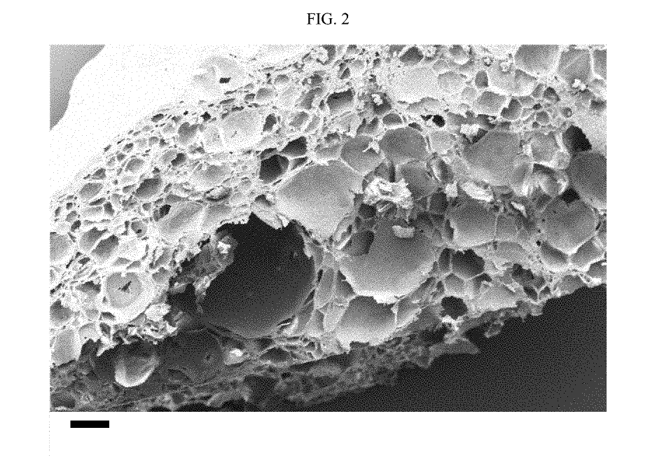

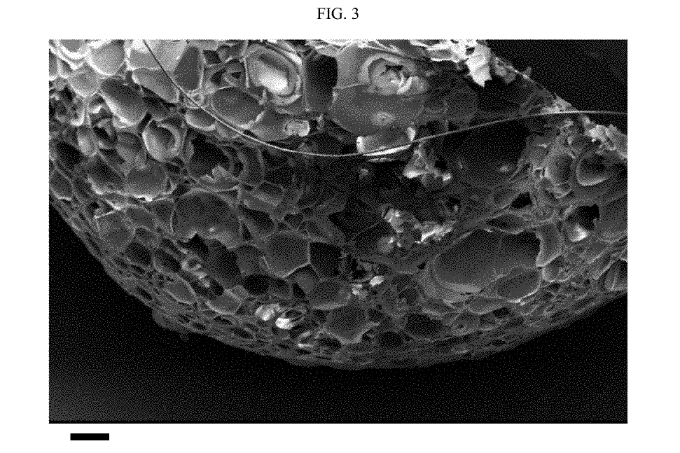

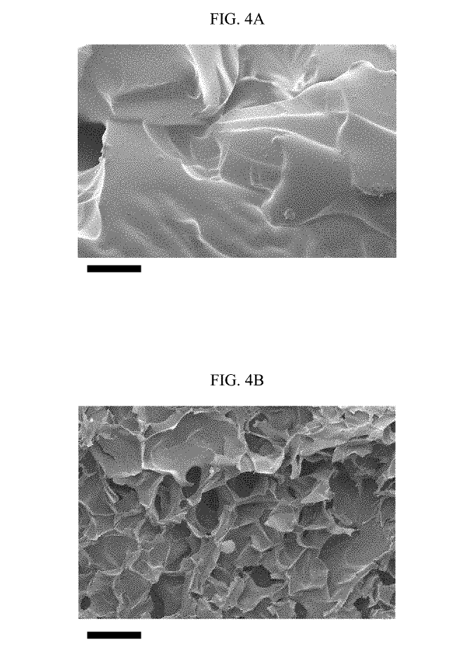

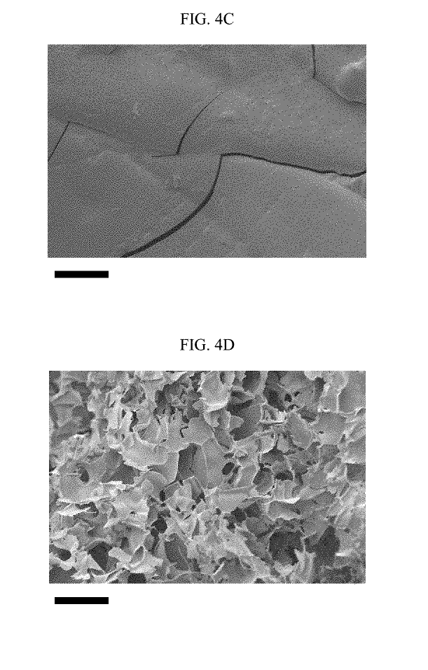

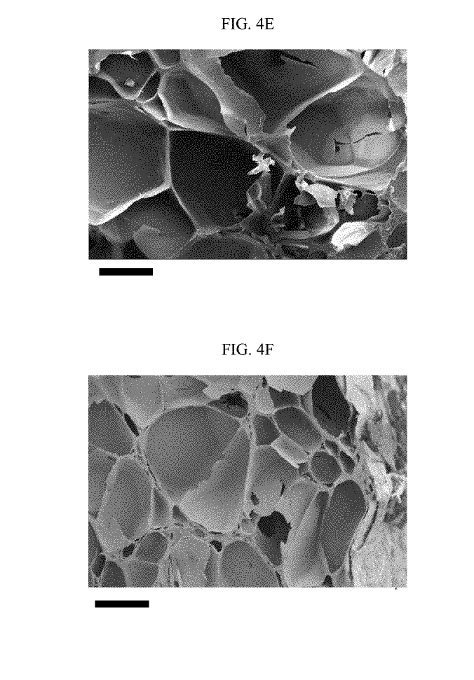

SEM images of the SF cryogels (FIG. 2) and SF hydrogels (FIG. 3) demonstrated that both structures were porous in nature; however, many of the pores appeared to be closed and in most cases not evenly distributed. The appearance of closed pores may possibly be the result of a sputter coating artifact. FIG. 4 shows comparisons between CG hydrogels (FIG. 4A), CG cryogels (FIG. 4B), NVP hydrogels (FIG. 4C), NVP cryogels (FIG. 4D), SF hydrogels (FIG. 4E), and SF cryogel (FIG. 4F).

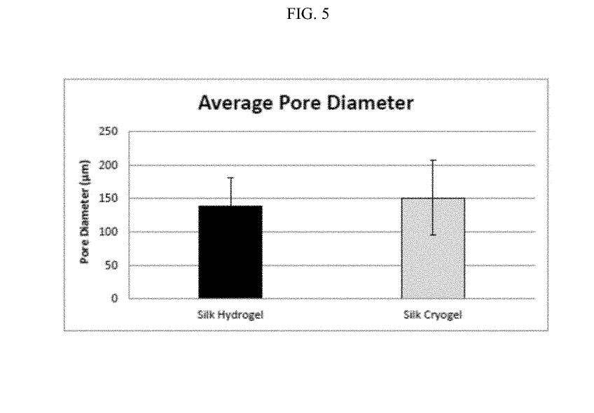

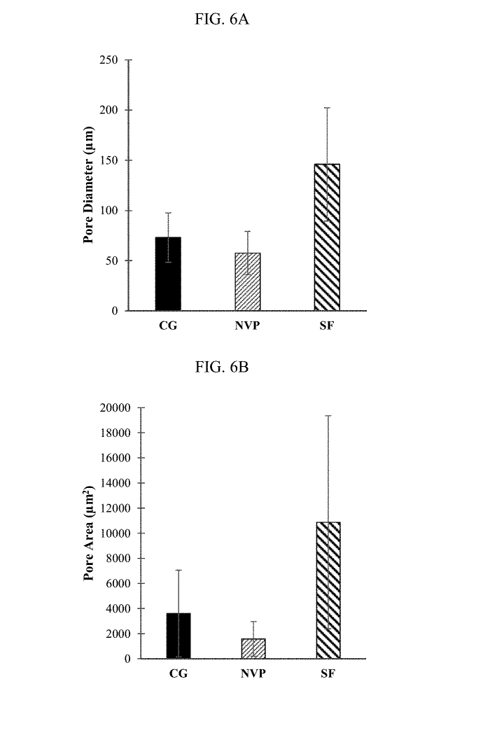

Pore diameters were measured from the SEM images using ImageJ. The SF hydrogel produced an average pore diameter of 138.92±42.67 μm, which was slightly smaller than the SF cryogel average of 150.77±55.96 μm (FIG. 5). As compared to CG and NVP cryogels, SF cryogels possessed both the largest pore diameter and area with average values of 146.03 μm² and 10,873.07 μm², respectively (FIGS. 6A and 6B). Previous literature has identified a pore diameter of at least 100 μm to be necessary for cellular infiltration and angiogenesis formation in bone applications. Thus, the diameter measurements of SF cryogels falls above the required threshold of 100 μm for bone applications. However, it should be noted that while SEM analysis provide a solid representation of the surface of a scaffold, it provides little insight into the structure's interior. Because ImageJ measurements are 2D representations of 3D structures, more advanced scaffold characterization techniques were employed.

### Example 4

In this Example, μCT of SF cryogels was conducted to evaluate pore size and interconnectivity.

To further evaluate pore size and interconnectivity a μCT (μCT 35, Scanco Medical, Wayne, Pa.; X-ray tube potential 45 kVp, integration time 600 ms, X-ray intensity 4 W, isotropic voxel size 7 μm, frame averaging 1, projections 500, medium resolution scan) was used. Three samples of each type of cryogel were scanned at thresholds 50, 60, 70, 80, 90, 100, and 110. A threshold of 80 was chosen to record measurements based upon user experience and pore clarity. The average pore diameter (um), scaffold connection density (1/mm³), and total ratio filled with scaffold were obtained.

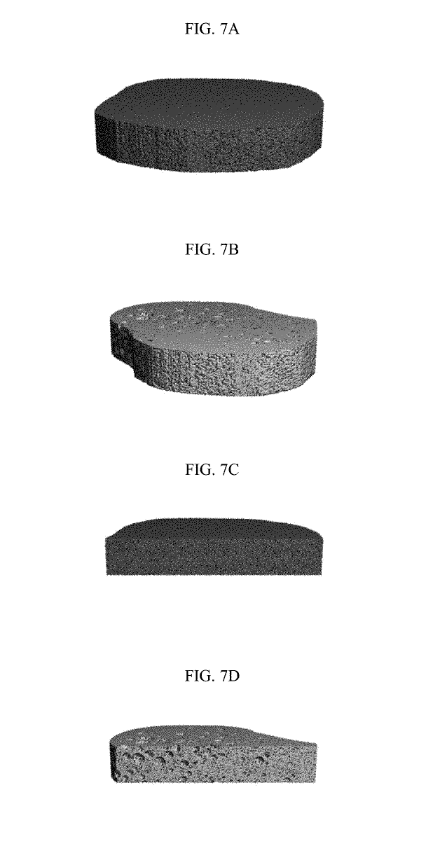

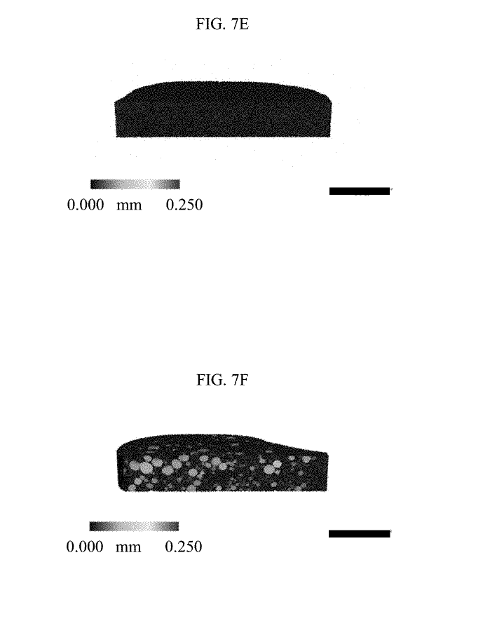

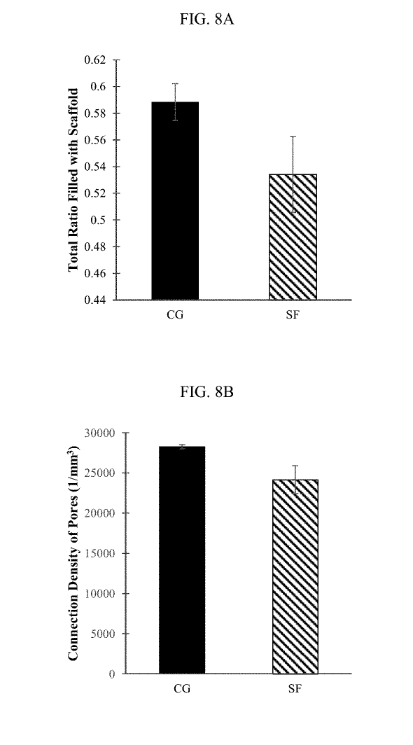

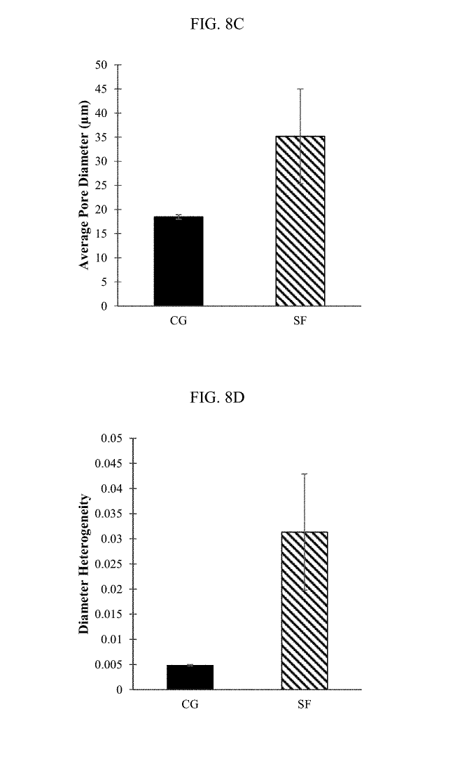

All types of cryogels were scanned with the μCT, but only CG cryogels and SF cryogels had the stability to be scanned by the μCT whereas the NVP cryogel fragmented when placed on the stand. Additionally, none of the hydrogel counterparts could be tested as their structure was not sturdy enough to fit on the stand and was composed of too much water. FIG. 7 shows 3D reconstruction images of the CG cryogels (FIG. 7A) and SF cryogels (FIG. 7B) and their porosity. CG cryogels had a small, even pore distribution (FIGS. 7C & 7E) and SF cryogels had a much more variable pore distribution (FIGS. 7D & 7F). Overall, 58.5% of the total volume of the scaffold was filled with CG material, and 53.42% with SF material (FIG. 8A). The μCT reported much lower average pore diameters of 18.47 μm and 35.17 μm for CG and SF cryogels, respectively (FIG. 8C). The heterogeneity of these diameters was much larger for SF, with a standard deviation of 0.031 as opposed to 0.005 for CG. This shows a much larger variation in pore size throughout the scaffold, as also shown in all other methods of pore diameter measurement (FIG. 8D). Additionally, the average connection density of the pores was reported at 28,238.70 l/mm³ for CG and 24,146.50 l/mm³ for SF cryogels (FIG. 8B). This data suggests that while SF has the largest pore diameter, CG cryogels possess a slightly larger pore interconnectivity. These data support the ImageJ measurements with SF cryogels having the largest diameter, but a much smaller value was found using this measurement technique.

### Example 5

In this Example, mercury intrusion porosimetry of SF cryogels was analyzed to evaluate the porosity of the different sample types.

A Quantachrome Instruments Ultrapyc 1200e pycnometer (model no. MUPY-31) was employed. Density analysis was completed according to manufacturer's protocol using ultrapure helium gas and a maximum pressure of 3 psig. For each sample, the sample weight was entered into the instrument's software and the pycnometer completed a total of 9 runs, averaging the 5 runs with the best standard deviations. A Thermo Scientific Pascal 140 Series porosimeter with elemental mercury (ALFA AESAR® 99.9% redistilled mercury) was used for the samples. The samples underwent pressurized mercury intrusion according to manufacturer's instrument protocol with the use of Dilatometer 44 (mercury height: 90.5 mm, stem mercury height: 64.5 mm, filling volume: 456 mm³, cone height: 21.0 mm, electrode gap: 5.0 mm, stem radius: 1.5 mm). The individual sample's weight and density (previously obtained via the pycnometer) were entered prior to mercury filling. After the sample was loaded into the dilatometer, the dilatometer was filled with mercury to its filling volume and then pressurized to the instrument's maximum pressure of 400 kPa. After completion of the mercury intrusion, data regarding the sample's porosity was collected and used in further sample analysis. The process was repeated for both dry and hydrated samples. The SF cryogel samples were hydrated in DI water for 48 hours prior to testing. The CG cryogel and NVP cryogel samples were hydrated in DI water for 10 minutes prior to resting. For the hydrated samples, the sample type's respective densities were maintained, but their hydrated weight was used as their respective sample weight. The porous nature of the hydrogels did not allow for testing using this procedure.

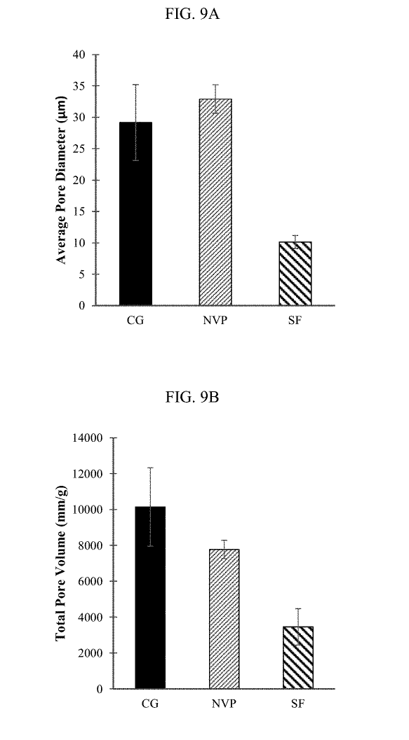

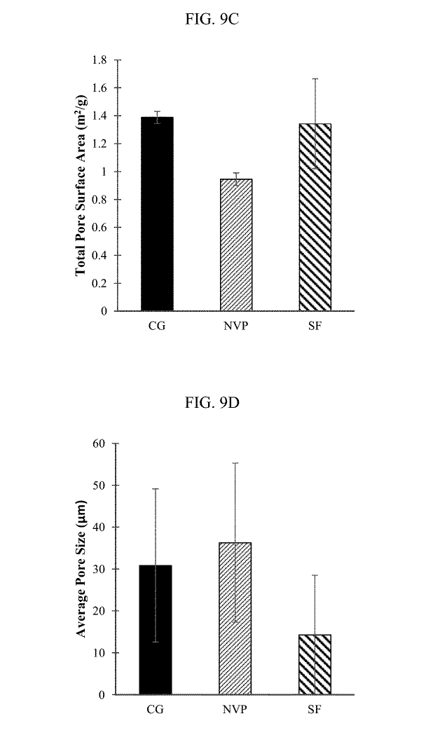

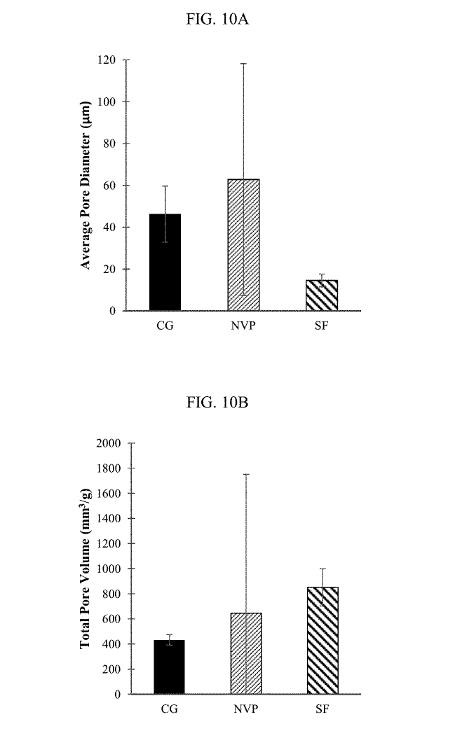

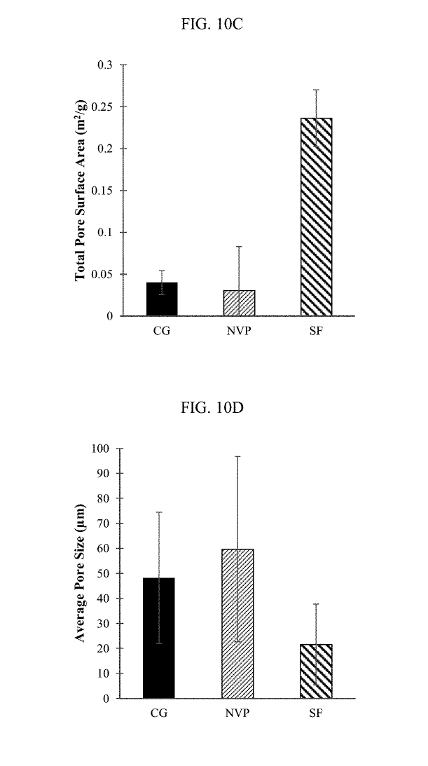

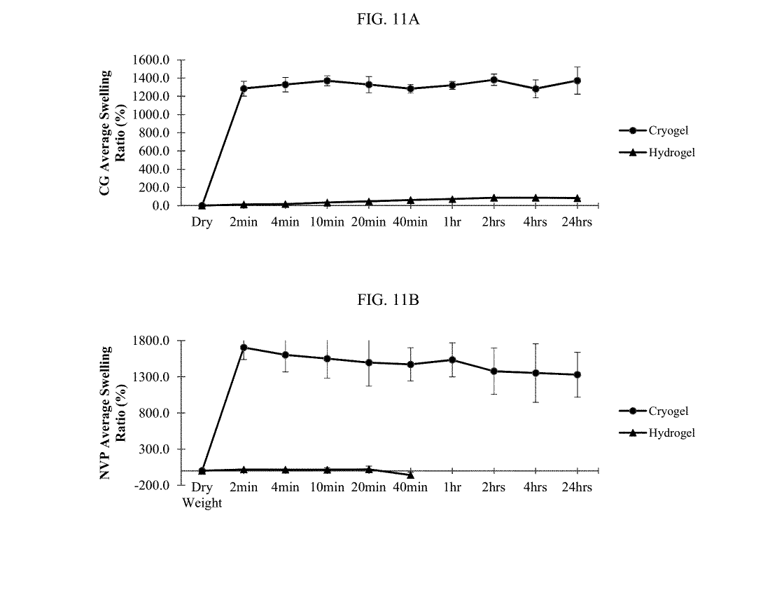

Mercury intrusion porosimetry was used as another method to analyze the various properties of the pores in the cryogels. Upon dehydration, NVP had the highest average pore diameter of 32.92 μm, followed by CG with 29.18 μm, and SF with 10.15 μm (FIG. 9A). The average pore diameter of the hydrated samples was highest for NVP with 62.83 μm, then CG with 46.27 μm, and lastly SF with 14.58 μm (FIG. 10A). All of these measurements being larger than the dry measurements. However, unlike the other pore measurement techniques, SF had the smallest and NVP the largest diameter compared with the other cryogels. Next, the complete volume of the pores was examined for the dry samples with CG possessing the largest value of 10,144.50 mm³/g, NVP with 7,770.99 mm³/g, and SF with 3,459.42 mm³/g (FIG. 9B). For the hydrated samples, SF had the largest volume of 850.60 mm³/g, followed by NVP with 644.85 mm³/g, and CG with 423.87 mm³/g. All of these sample values are much smaller than the dry samples (FIG. 10B). Mercury porosimetry also provided the total pore surface area which, for the dry samples, was 1.39 m²/g for CG, 0.95 m²/g for NVP, and 1.34 m²/g for SF (FIG. 9C). For the hydrated samples, SF had the largest volume of 0.24 m²/g, followed by CG at 0.04 m²/g, and NVP at 0.03 m²/g, all of which are smaller than the dry samples (FIG. 10C). Lastly, the average pore size was also reported to be compared to previous methods of measurement. Here, there was an average pore size of 30.84 μm for CG, 36.28 μm for NVP, and 14.30 μm for SF (FIG. 5D). The hydrated samples pore size were 26.20 μm for CG, 37.04 μm for NVP, and 16.16 um for SF (FIG. 10D).

### Example 6

In this Example, swelling properties of SF cryogels were analyzed for shape retention and rehydration potential of the constructs.

Three samples of each hydrogel and cryogel were completely dehydrated for 48 hours. After being placed in DI water, each sample was removed and weighed at time points 2 minutes, 4 minutes, 10 minutes, 20 minutes, 40 minutes, 1 hour, 2 hours, 4 hours, and 24 hours. The average swelling ratio was calculated according to equation (1) using the dry weight (W_{d}) and the swollen weight (Wₛ):

Swelling Ratio=(Wₛ−W_{d})/(W_{d})

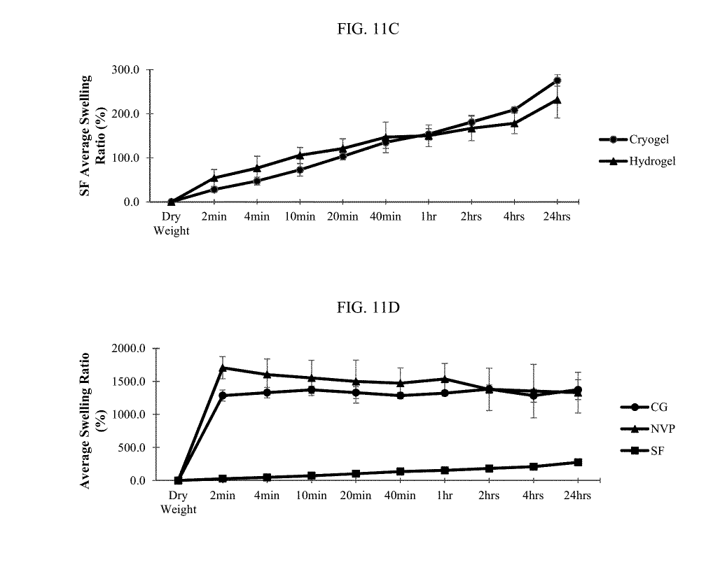

All cryogels swelled to at least 275% of their original dry weight (FIGS. 11A-11C). The CG and NVP hydrogels demonstrated minimal amounts of swelling (FIGS. 11A & 11B), however the SF hydrogel showed similar swelling ability to the SF cryogel (FIG. 11C). By 40 minutes, the NVP hydrogels had broken down so drastically that a negative average swelling ratio (%) was recorded and after this time point, no further data could be collected (FIG. 11B). This shows a general superiority of cryogels to hydrogels for swelling upon rehydration to obtain their original morphology. When plotted against one another, CG and NVP cryogels reached their maximum swelling potential after two hours whereas SF cryogels reached their lower swelling potential after 24 hours (FIG. 11D).

### Example 7

In this Example, ultimate compression of SF cryogel was conducted to analyze the mechanical integrity of the hydrogels and cryogels.

Ultimate compression at both 50% and 80% was completed for each material type (n=6) using a Mechanical Testing System (MTS Criterion Model 42, MTS Systems Corporation) was fitted with a 100 N load cell. A test rate of 10 mm/min, preload of 0.05 N, data acquisition rate of 10 Hz, and preload speed of 1 mm/min was used to compress each sample to either 50 or 80% of its original volume, taking into account both the diameter and thickness. Data integration was completed using MTS TW Elite software to record both the peak stress (kPa) and modulus (kPa).

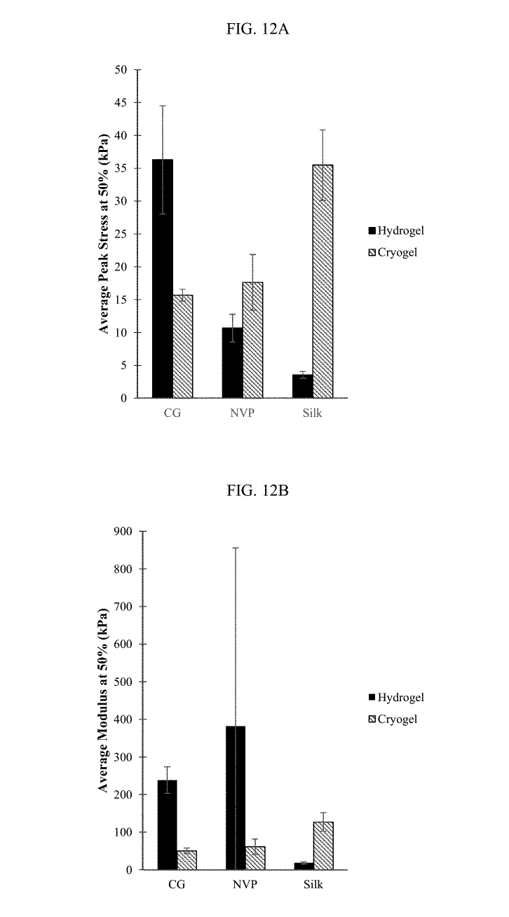

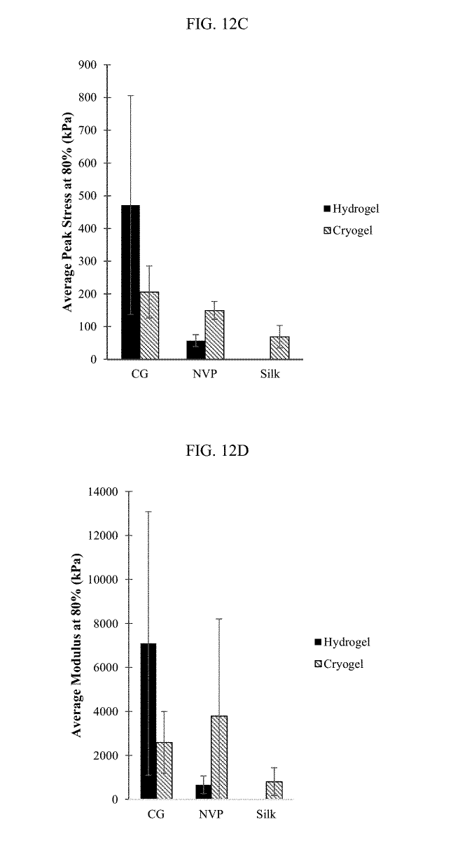

At both the 50% and 80% strains, the CG hydrogels and SF cryogels at 50% had the highest average peak stress showing their strength (FIGS. 12A & 12C). All hydrogels other than SF had a higher average modulus than the cryogels at 50% demonstrating the materials stiffness (FIG. 12B). At 80% ultimate compression, the NVP cryogel exhibited a higher modulus than its hydrogel counterpart, but CG hydrogels were still higher than CG cryogels (FIG. 12D). Additionally, SF hydrogels were not tested at 80% due to their complete loss of mechanical integrity at 50% compression. Since the hydrogels are largely composed of water the structures were able to withstand high loads directly applied to the object, but then failed mechanically. By comparison, the spongy structure of the cryogels did not show as much resistance to compression, and allowed for the materials to return to their original shape when the load was removed.

### Example 8

In this Example, compressive cyclic loading with degradation of SF cryogels was conducted to compare the hydrogels and cryogels ability to withstand repeated application of a load and overall hysteresis.

Five samples of each type of hydrogel and cryogel were cyclically loaded 20 times using the MTS system described in Example 7 and then placed in sterile phosphate buffered saline (PBS). The samples underwent cyclic loading on days 1, 3, 7, 14, 21, and 28 and placed in fresh PBS after each test. Cyclic loading parameters included a preload speed of 2.54 mm/min, plate separation force of 4.448 N, test speed of 10 mm/min, plate separation speed of 10 mm/min, hold times of 0 seconds, preload of 0.05 N, and compression of 20% and 5%. Data integration was completed using MTS TW Elite software and the percent stress-relaxation and hysteresis were found using a premade Matlab program.

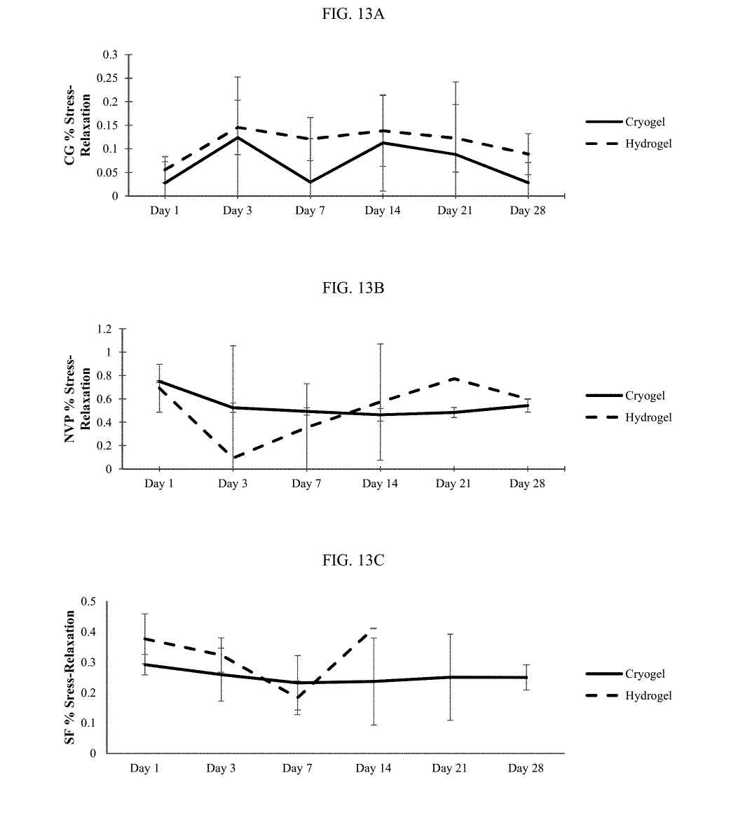

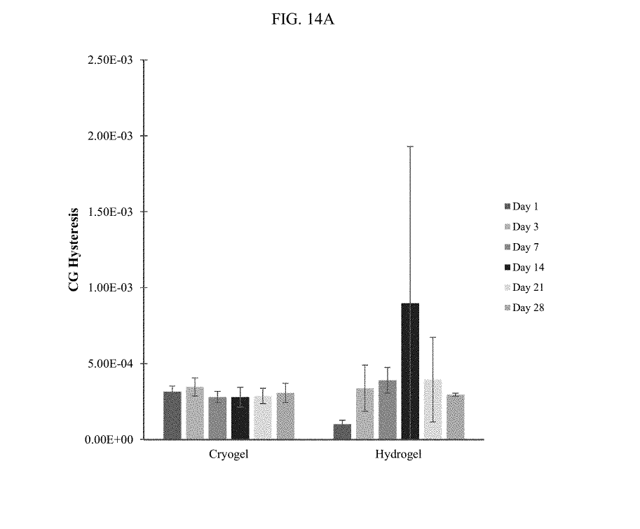

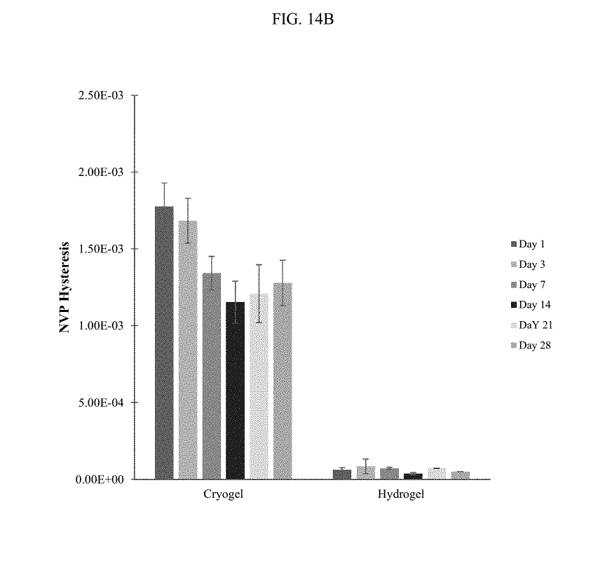

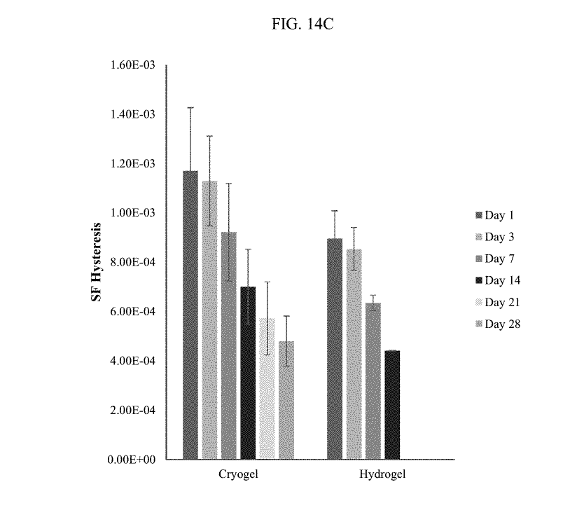

The percent stress-relaxation of each hydrogel and cryogel was recorded, providing further information on the overall change in structure. Higher values denote a larger deformation of the sample, demonstrating decreased resilience. All cryogels showed a lower percent stress-relaxation compared to their hydrogel counterparts (FIGS. 13A-13C). The SF hydrogels were completely fragmented after Day 14 (FIG. 13C). Additionally, the CG hydrogels reduced their thickness by approximately 25% thus becoming denser over the 28 days (FIG. 13A). This allowed the CG hydrogels to withstand the cyclic loading better than expected and thus, was not an accurate representation of CG hydrogel stress-relaxation and hysteresis. Hysteresis, or the loss of energy through loading and unloading, shows how well the structures were able to maintain their mechanical integrity over multiple load applications. The CG cryogels had a very low, constant hysteresis in comparison to the hydrogels (FIG. 14A). The NVP hydrogels showed a superior hysteresis to the NVP cryogels (FIG. 14B) and the SF hydrogels and cryogels had very similar hysteresis (FIG. 14C). Overall, the SF hydrogels were completely fractured by Day 14 and NVP hydrogels crumbled and did not hold their original shape. While NVP hydrogels showed superior hysteresis, they were not actually experiencing cyclic loading accurately. Additionally, all cryogels lasted the full 28 days and maintained their original shape and integrity, even with PBS degradation.

### Example 9

In this Example, acellular mineralization of SF cryogel was conducted.

All cryogels were sterilized in 70% ethanol (Fisher Scientific, NJ) on a shaker plate for 30 minutes, followed by an additional 30 minutes in 70% ethanol in the fume hood, and three 10 minute washes with sterile PBS. Half of the scaffolds were then soaked in complete media composed of Dulbecco's Modified Eagle's Medium (DMEM) with 4.5 g/L Glucose & L-Glutamine (Lonza, Md.), 10% fetal bovine serum (FBS) (Biowest, Tex.) and 1% penicillin-streptomycin solution (Hyclone, Pa.) for an additional hour to allow for protein absorption and potentially enhanced cellular attachment. Once sterilized, all scaffolds were placed in a 48 well plate (Falcon, N.Y.). 100 μL of media containing 50,000 human bone osteosarcoma-derived cells (MG-63; ATCC, VA) were seeded onto each scaffold by slowly dripping the solution on the top. Once seeded, the 48 well plates were incubated for two hours at 37° C. and 5% CO₂ to allow the attachment of the cells. At this time an additional 175 μL of complete media was added so that all samples were completely submerged. The media was changed every two to three days from around the scaffold. The cryogels were removed at days 7, 14, 21, and 28 and placed in formalin (Protocol, Mich.). Half of each scaffold was embedded in paraffin and sectioned using a microtome. These sections were then stained with DAPI to observe cellular infiltration over the various time points. The other half of the scaffolds from days 7, 14, 21, and 28 were stained with alizarin red to detect any presence of mineralization. Sections of the scaffold were also SEM imaged to detect any surface and internal mineralization. The procedure to make c-SBF was adopted from Oyane et al. (J. Biomed. Mater. 2003, 65A(2):188-195) and the chemical composition to make 100 mL of c-SBF summarized in Table 3:

TABLE 3
Chemical composition of c-SBF.

| Chemical | Amount | |
|---|---|---|
| NaCl | 0.8036 | g |
| NaHCO₃ | 0.0352 | g |
| KCl | 0.0225 | g |
| K₂HPO₄ · 3H₂O | 0.0230 | g |
| MgCl₂ | 0.0146 | g |
| 1.0M HCl | 4 | mL |
| CaCl₂ | 0.0293 | g |
| Na₂SO₄ | 0.0072 | g |
| TRIS | 0.6063 | g |

For each condition and at each time, three samples were used to quantify mineralization using an Alizarin Red S (ARS) staining procedure, three samples underwent ultimate uniaxial compressive tests at a strain end point of 50% (see above), and one sample was dried at room temperature for scanning electron microscope imaging (see above). In the assay, three samples of each condition that were not in c-SBF were also tested for control purposes. Absorbance readings for the ARS assay were analyzed with a SpectraMax i3 spectrophotometer.

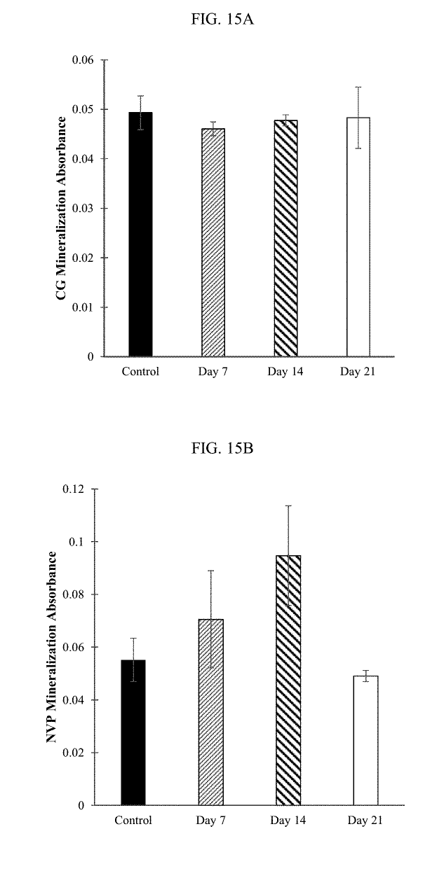

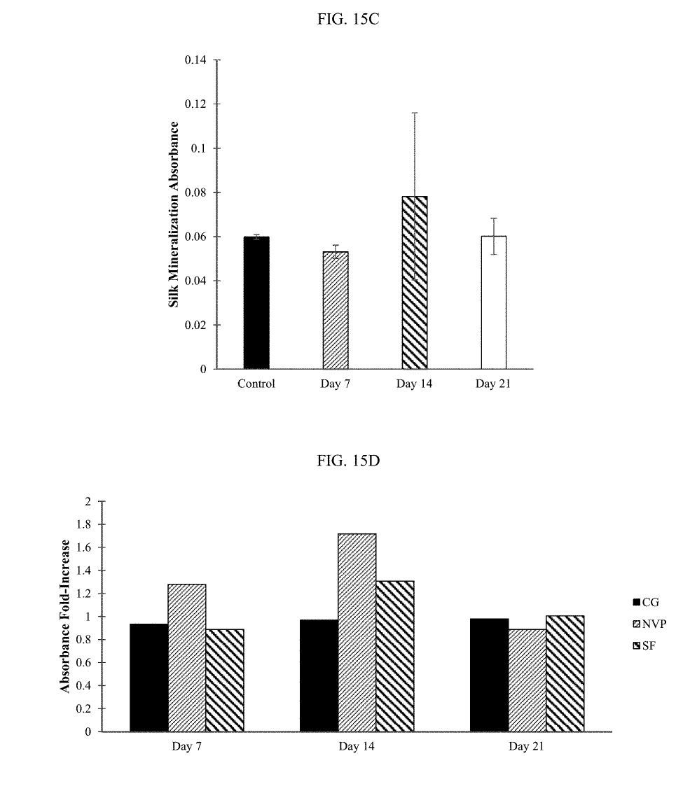

Upon mineralization for 7, 14, and 21 days, the acellular cryogel samples were stained with alizarin red stain (ARS) and absorbance was measured at 550 nm. The CG cryogels did not show any change in mineralization levels over 21 days (FIG. 15A). NVP and SF cryogels showed a slight increase in mineralization through day 14 and then a drop in absorbance levels (FIGS. 15B & 15C). The samples became so weak by day 21 that their fragmentation made it very difficult to accurately measure absorbance. The fold increase was calculated using the control as the initial value and plotted for all cryogels (FIG. 15D). When plotted on a single graph, it can be seen that all cryogels had essentially negligible mineralization over 21 days compared to the control materials.

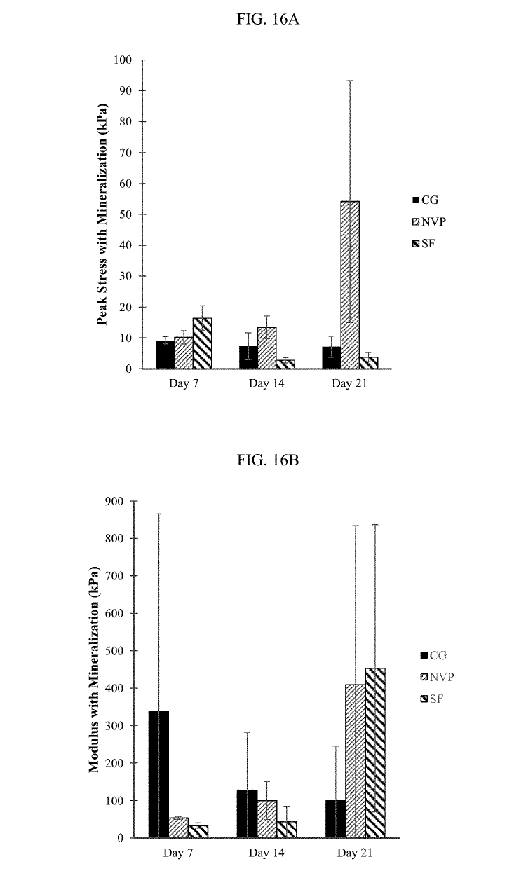

Ultimate compression at 50% was done on each type of cryogel (n=3) as shown in FIGS. 16A & 16B. CG cryogels had a fairly constant peak stress over all time points, supporting the previous data that these cryogels were not undergoing any sort of mineralization (FIG. 16A). NVP cryogels peak stress increased over the 21 days, while the SF cryogels decreased after only a week. The SF cryogels experienced some fragmentation which made it difficult to complete ultimate compression (FIG. 16A). Both NVP and SF cryogels increased their modulus over 21 days, suggesting a small amount of mineralization may have occurred and a corresponding increase in strength existed (FIG. 16B).

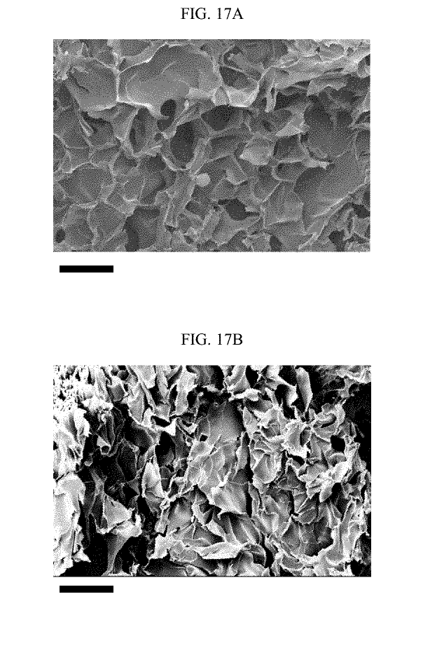

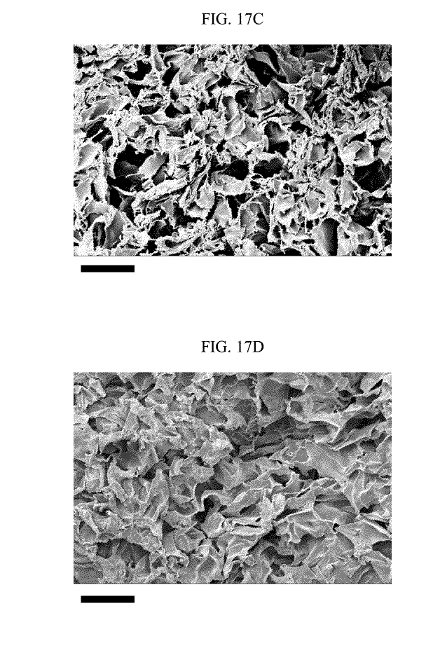

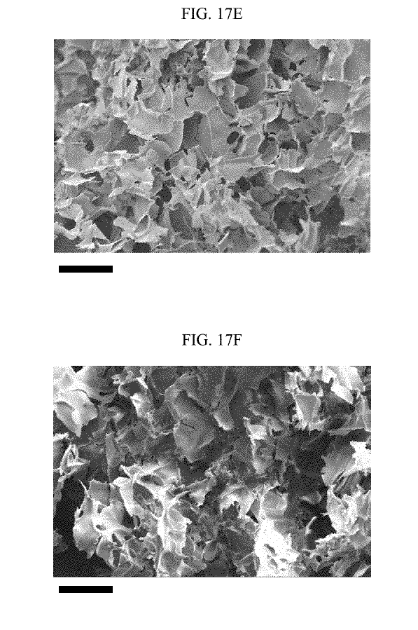

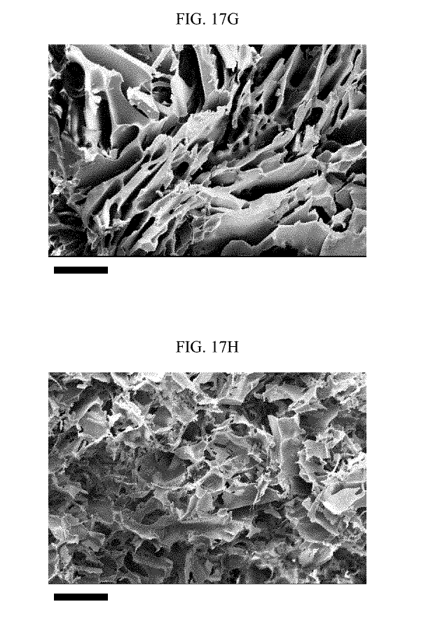

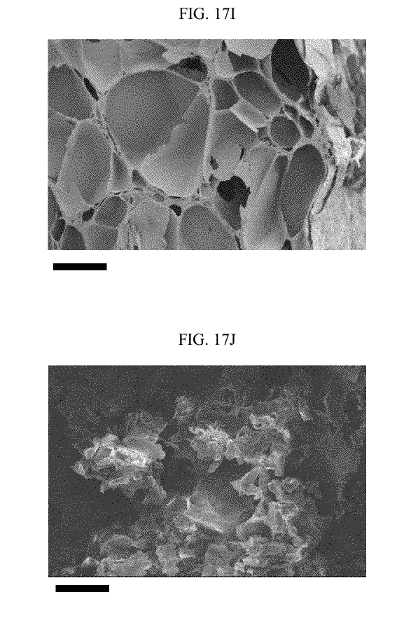

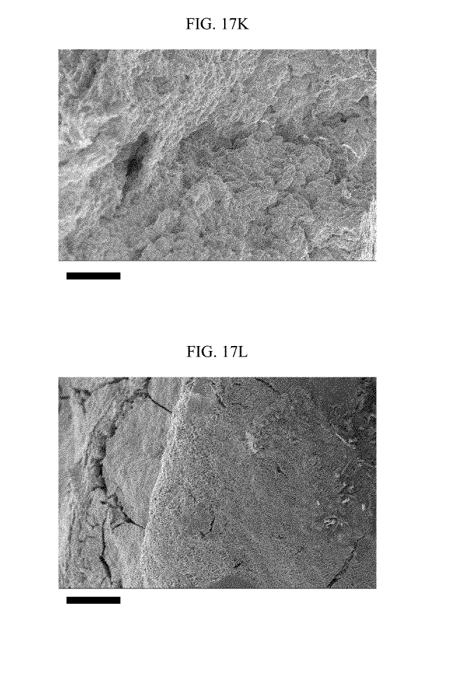

SEM images were obtained for each cryogel mineralized over 7, 14, and 21 days. By day 14, all cryogels showed a small amount of mineralization and once day 21 was reached, there was substantial mineralization on all cryogel types (see, FIGS. 17A-17L).

These results demonstrated that the methods of the present disclosure can be used to form macroporous silk fibroin cryogels via sonication induced (3-sheet formation. The SF cryogels were found to be more mechanically stable then their hydrogel counterparts in both uniaxial compression testing and cyclic loading. The addition of bone char increased the mineralization potential of the cellularized fibroin scaffolds.

The methods can be used to prepare silk fibroin cryogels that are useful for tissue engineering. The incorporation of materials such as biomolecules, additives and cells allow for specific tissue engineering purposes such as bone repair and regeneration.

What is claimed is:

1. A composition comprising a silk fibroin cryogel.

2. The composition of claim 1, further comprising at least one biomolecule.

3. The composition of claim 2, wherein the at least one biomolecule is selected from the group consisting of a growth factor, a cytokine, a bioactive lipid, an immunoglobulin, and combinations thereof.

4. The composition of claim 3, wherein the at least one biomolecule is a preparation rich in growth factors.

5. The composition of claim 1, further comprising at least one cell adhesion molecule.

6. The composition of claim 5, wherein the cell adhesion molecule is selected from the group consisting of fibronectin, vitronectin, collagen, an RGD (arginine-glycine-aspartic acid) peptide, a LDV (leucine-aspartic acid-valine) peptide, laminin and combinations thereof.

7. The composition of claim 1, comprising from about 1% (w/v) to about 10% (w/v) silk fibroin protein.

8. The composition of claim 1, comprising a pore diameter ranging from about 15 um to about 500 μm.

9. The composition of claim 1, comprising a pore area ranging from about 2,000 μm² to about 20,000 μm².

10. The composition of claim 1, further comprising at least one additive.

11. The composition of claim 1, further comprising a cell.

12. A method for preparing a silk fibroin cryogel, the method comprising isolating silk fibroin to prepare a silk fibroin solution; sonicating the silk fibroin protein solution; and subjecting the sonicated silk fibroin solution to at least one freeze-thaw cycle.

13. The method of claim 12, further comprising adding at least one biomolecule to the silk fibroin solution.

14. The method of claim 13, wherein the at least one biomolecule is selected from the group consisting of a growth factor, a cytokine, a bioactive lipid, an immunoglobulin, and combinations thereof.

15. The method of claim 12, wherein the at least one biomolecule is a preparation rich in growth factors.

16. The method of claim 12, further comprising adding at least one cell adhesion molecule to the silk fibroin solution.

17. The method of claim 16, wherein the cell adhesion molecule is selected from the group consisting of fibronectin, vitronectin, collagen, an RGD (arginine-glycine-aspartic acid) peptide, a LDV (leucine-aspartic acid-valine) peptide, laminin and combinations thereof.

18. The method of claim 12, further comprising adding at least one additive to the silk fibroin solution.

19. The method of claim 18, wherein the at least one additive is selected from the group consisting of an osteoinductive agent, an osteoconductive agent, and combinations thereof.
